(19) United States
(12) Patent Application Publication
(10) Pub. No.: US 2022/0202601 A1
(43) Pub. Date: Jun. 30, 2022

(54) METHODS AND DEVICES FOR TREATING ILIOCAVAL COMPRESSION, OCCLUSION, REDUCTION OF VENOUS CALIBER, AND SYNDROMES AND DISEASE STATES RESULTING FROM OCCLUSION

(71) Applicant: DP Holding (U.K) Limited, Grantham (GB)

(72) Inventors: Darren SPENCER, Oxford (GB); Peter BALMFORTH, Oslo (GB); Paul SOBOTKA, Minneapolis, MN (US); Rodney BRENNEMAN, San Diego, CA (US)

(21) Appl. No.: 17/609,324

(22) PCT Filed: May 7, 2020
(86) PCT No.: PCT/US2020/031908

§ 371 Date: Nov. 5, 2021

Related U.S. Application Data
(60) Provisional application No. 62/844,994, filed on May 8, 2019.
(60) Provisional application No. 62/903,354, filed on Sep. 20, 2019.

(51) Int. Cl. A61F 2/90; D04C 1/06; A61F 2/95; D04C 1/02

(52) U.S. Cl. CPC A61F 2/90; D04C 1/06; A61F 2/95; D04C 1/02; A61F 2230/0067; D10B 2101/20; A61F 2250/0039; A61F 2250/0048; A61F 2250/0098; D10B 2509/06; A61F 2250/0023

(57) ABSTRACT

Aspects of the invention provide a self-expanding stent device. The device comprises: an elongated body that defines a lumen within. The body has a first and a second terminus and a longitudinal axis located therebetween. The body comprises a first zone and a second zone along the longitudinal axis. When the device is in an expanded configuration the first zone has a first radial strength that is resistant to an external compressive force, and the second zone has a second radial strength that is resistant to an external compressive force. The radial strength of the first zone is greater than that of the second zone. The diameter of the body lumen proximal to the first terminus is greater than that of the lumen proximal to the second terminus.

### TECHNICAL FIELD

The present invention relates to methods and devices for treating atrial fibrillation, hypertension, erectile dysfunction, venous ulcers, syncope, dysmenorrhea, deep vein thrombosis and heart failure with preserved ejection fraction and heart failure with reduced ejection fraction as well as a plurality of other diseases associated with reduction or interference of venous return and more generally with cardiovascular or circulatory dysfunction, including May-Thurner syndrome.

### BACKGROUND

Venous congestion is most often considered to be a consequence of cardiac congestion or total body volume overload. However, veins may become congested due to local embarrassment of the free flow of blood returning to the heart. The role of veins per se as neurohormonally active structures and participants in disorders such as heart failure is seldom considered. Indeed, when a patient presents with symptoms of heart failure they will conventionally be referred to a cardiologist who will naturally focus their investigations on the anatomy and physiology of the patient's heart, without consideration of potential contributions of pelvic venous obstructions to the signs and symptoms of exertional intolerance and lower extremity congestion.

The intravascular correction of venous disorders in the pelvis is achieved using devices largely designed for use elsewhere in the body, such as legs or arms, which are not ideal for the anatomy and pathology unique to pelvic venous disorders. Often the pelvic stenting of diseased veins is carried out using two stents placed in the region end to end. However, quite often in this technique a gap exists whereby it is common for the area between the stents to develop a restenosis. Ideally, stents are designed to treat obstructions of known locations and the stent characteristics have been designed for that specific location and obstruction type. In reality, the location of culprit occlusions and external compressions causing obstructions to flow or changes in caliber are unknown. In order to achieve optimal and uncompromised results, stents addressing pelvic obstructions must adapt to the needs of both internal obstructions and those caused by external compressions as well as the unique flexion points required by pelvic venous anatomy.

It is an object of the invention to provide devices and methods that address at least some of the disadvantages associated with the prior art.

### SUMMARY

The present invention relates to the surprising finding that iliocaval venous compression, occlusion, reduction of caliber and/or reduction of venous return results in a cascade of previously-thought unrelated syndromes, diseases and disease states. The implantation of a device within the iliocaval region of the body can alleviate these compressions, occlusions, reductions of caliber and/or venous return and consequently can treat the cascade of syndromes, diseases, and disease states.

In particular, but not exclusively, aspects of the invention relate to a method of treating atrial fibrillation in a patient, a method of treating hypertension in a patient, a method of treating erectile dysfunction in a patient, a method of treating venous ulcers in a patient, a method of treating syncope in a patient, a method of treating deep vein thrombosis in a patient and to a method of treating heart failure with preserved ejection fraction, and heart failure with reduced ejection fraction in a patient.

Aspects of the invention also provide a self-expanding stent device. The stent device comprises a woven or braided elongate body that defines a lumen within, the body having at least a first and at least a second terminus and a longitudinal axis located therebetween; wherein the body comprises at least a first zone and at least a second zone along the longitudinal axis; wherein when the device is in an expanded configuration the first zone has a first radial strength that is resistant to an external compressive force, and the second zone has a second radial strength that is resistant to an external compressive force, wherein the radial strength of the first zone is greater than that of the second zone; and wherein the diameter of the body lumen at or proximal to the first terminus is greater than that of the lumen at or proximal to the second terminus. In an embodiment, the body may be substantially cylindrical or flattened cylindrical in configuration for all or a part of the body. The stent device may comprise a plurality of sections joined together to form the lumen.

Advantageously, the zones of varying radial strength overcome at least some of the disadvantages associated with the prior art including foreshortening, lack of flexibility and vessel wear.

Correspondingly, the inventive concept embraces a system for deployment of a venous stent, the system comprises a delivery catheter and a self-expanding stent as described in embodiments herein.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### DRAWINGS

One or more embodiments of the invention will now be described, by way of example only, with reference to the accompanying drawings, in which:

FIG. 1 shows an anatomical description of the main pelvic arteries and veins.

FIG. 2 shows an anatomical description of the iliocaval junction veins (the external iliac vein through to the inferior vena cava) in the pelvic region.

FIGS. 3(a) and 3(b) show different perspectives of the iliocaval junction highlighting the vessels that are unsupported by skeletal muscle.

FIG. 4 shows a graph illustrating the systolic, diastolic, mean arterial and pulse pressures throughout the blood vessels.

FIG. 5 shows an MRI image of the iliocaval region of a patient exhibiting persistent and drug refractory hypertension. The image shows the presence of a stenosis of the external iliac vein.

FIG. 6 shows the general procedure of screening that should be applied for a patient presenting with any of the aforementioned symptoms, diseases and disease-states.

FIG. 7 shows a sample choice of antithrombotic regimens employed in the administration of drug based therapies used in addition to surgical treatments for obstruction to venous flow in the iliocaval region (DOAC=direct oral anticoagulant; LMWH=low molecular weight heparin).

FIG. 8 shows the key stent features to be considered in stent design, including chronic outward force, crush resistance and radial resistive force.

FIG. 9 shows the hoop strength (radial force) vs diameter of different stent design types, highlighting the increased radial resistive force of the hybrid stent design type.

FIG. 10 shows the optimal post stent diameter and area in iliac vein stenting.

FIG. 11 shows an asymmetric stent design shown as a braided construction, whereby the HCS (higher compressive strength) zone is formed by a tighter weave pattern and the LCS (lower compressive strength) zones are formed with a more open weave pattern.

FIG. 12 shows an asymmetric stent design shown as a braided construction, whereby the HCS zone is much longer in length relative to the length of the LCS zones.

FIG. 13 shows an example of asymmetric taper of the stent with the compressive strength of the different zones of the stent relative to the typical impingement/compression locations of both the right and left inferior vena cava.

FIG. 14 shows an example of single long venous stents with low compressive strength zones, a higher compressive strength zone in the intermediate of the stent and flexible termini. The embodiment shows tapered expanded diameters.

FIG. 15 shows an example of a stent with braided mesh design with additional radial compressive strengtheners present at one or more locations along the length of the stent.

FIG. 16 shows different examples, (a), (b), and (c) of the configuration of the reinforcing elements.

FIG. 17 shows a top view (a) and a side view (b) of an example whereby the base braided system further comprises anchor/coupling elements.

FIG. 18 shows an example whereby the anchor/coupling elements extend into the base braid system to provide radial reinforcement.

FIG. 19 shows an example of how an anchor/coupling element can be formed from the base braid system (a) or from the reinforcing element (b).

FIG. 20 shows a stent device for use in the iliocaval region according to an embodiment of the invention.

FIG. 21 shows an example of a stent having an inflow booster.

FIG. 22 shows an example of the stent of FIG. 21 placed in a vein adjacent an artery.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to setting forth the invention, a number of definitions are provided that will assist in the understanding of the invention.

As used in this description, the singular forms ‘a,’ ‘an,’ and ‘the’ include plural referents unless the context clearly dictates otherwise. Thus, for example, the term ‘a sensor’ is intended to mean a single sensor or more than one sensor or to an array of sensors. For the purposes of this specification, terms such as lorward,“rearward,”front,“back,”right,“left,” ‘upwardly,’ downwardly,' and the like are words of convenience and are not to be construed as limiting terms. Additionally, any reference referred to as being ‘incorporated herein’ is to be understood as being incorporated in its entirety.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘sympathetic nervous system’ (SNS) refers to one of two divisions of the autonomic nervous system, the other being the parasympathetic nervous system. The SNS operates through a series of interconnected neurons. The SNS enables the effective response to both internal and external circumstances. It is known to be the effector of neurogenic control of vascular tone, blood pressure, and heart rhythm and rate.

The term ‘sympathetic activity’ refers to the acute alteration of arterial muscle tone as well as the recruitment of venous volume into central circulation. These changes in sympathetic activity are generally acute and in response to the need to preserve systemic blood pressure and organ perfusion in response to trauma, stress, fight or flight response or postural changes, for example. However, the chronic elevation of sympathetic tone, in response to venous hypertension, results in a maladaptive process that can result in organ dysfunction.

Blood pressure is in part regulated through the maintenance of so-called ‘sympathetic tone’.

The SNS is activated by the vasomotor centre which results in a practically body wide modulation of the heart and both the veins and arterioles of a variety of tissues. This occurrence results in an overall increase in the systemic arterial pressure. Resting systemic arterial pressure is largely reliant on baseline SNS tone. SNS fibres are known to release norepinephrine on both arteriolar smooth muscle and venous vascular smooth muscle. As a consequence, both arteriolar and venous constriction can result.

The term ‘vascular tone’ refers to the level of constriction smooth muscle in a blood vessel experiences, relative to when in its fully dilated state. Vascular tone is determined by the balance of competing vasoconstrictor and vasodilator influences upon the vessel. Furthermore, the term ‘sympathetic tone’ refers to the condition of vascular smooth muscle when the tone is maintained predominantly by impulses from the sympathetic nervous system.

The term ‘venous obstruction’ includes, at least: venous stenosis, venous congestion, and venous constriction. It refers to any occurrence whereby the diameter (or ‘caliber’) of a vein is reduced when compared to a normal, i.e. non-occluded, state. Venous obstruction can occur through the narrowing (stenosis) of the vein, through blockage or through externally applied pressure causing a localised compression of the vein. The term also includes venous occlusion, whereby the vein's lumen is partially or totally obstructed to the flow of blood. Occlusion may result from thrombosis (e.g. deep vein thrombosis (DVT)) or may be due to tumour incursion. The term iliocaval venous obstruction' refers to a condition of the systemic veins of the abdomen. Overall, this results in a reduction in venous caliber and in alterations of venous pressure and blood return to the heart.

The term ‘venous return’ is defined by the volume of blood returning to the heart via the venous system, and is driven by the pressure gradient between the mean systemic pressure in the peripheral venous system and the mean right atrial pressure of the heart. This venous return determines the degree of stretch of heart muscle during filling, preload and is a major determinant of cardiac stroke volume.

The term ‘venous compression’ refers to the external compression of the vein. The source of external compression may be caused by an adjacently located artery compressing the vein against another fixed anatomical structure, which can include the bony or ligamentous structures found in the pelvis, the spine itself, or overlapping arterial branches.

The term ‘May-Thurnersyndronne’ (MTS) also known as iliac venous compression syndrome (which includes Cockett's syndrome) is a form of iliocaval venous compression wherein the left common iliac vein is compressed between the overlying right common iliac artery anteriorly and the lumbosacral spine posteriorly (fifth lumbar vertebra). Compression of the iliac vein may cause a myriad of adverse effects, including, but not limited to discomfort, swelling and pain.

The compression of the iliac vein and reduction in venous flow in some cases can result in flow stasis causing DVT (Deep Vein Thrombosis). DVT refers to a medical condition wherein a blood clot (thrombus) forms in a vein. This is most commonly found in the leg. One of the major contributing factors to the formation of a clot is the pooling of venous blood. The presence of prolonged venous engorgement and blood flow stasis are conditions of risk for developing deep vein thrombosis. Treatment of the obstructing lesion is critical in relieving the underlying conditions for thrombosis and protecting against recurrence. Other less common variations of May-Thurner syndrome have been described such as compression of the right common iliac vein by the right common iliac artery; this is known as Cockett's syndrome. More recently, the definition of May-Thurner syndrome has been expanded to include an array of compression disorders associated with discomfort, leg swelling and pain, without the manifestation of a thrombus. Collectively, this has been termed non-thrombotic iliac vein lesions (NIVL).

The course that the left common iliac vein takes is less direct than that of the right common iliac vein which extends generally parallel to the inferior vena cava. Along this course it lies under the right common iliac artery, which may cause it to compress against the lumbar spine. Iliac vein compression is a frequent anatomic variant. It is possible for an individual to not present any outward signs of swelling, pain or thrombosis in the leg. Compression of the left common iliac vein becomes clinically significant only if such compression causes appreciable hemodynamic changes in venous flow or venous pressure, or if it leads to acute deep venous thrombosis. When the system is stressed e.g. during exercise it is common for the embarrassment of venous flow to be evident by the heightened demands of lower extremity blood flow during exercise. In addition to the other problems associated with compression and embarrassed venous blood flow return, the vein may also develop intraluminal fibrous spurs from the effects of chronic pulsatile compressive forces from the overlying artery.

The term ‘intraluminal thickening’ (also referred to as venous spurs or intraluminal spurs) is related to this external compression of the left common iliac vein by the right common iliac artery against the fifth lumbar vertebra. Venous spurs arise due to the chronic pulsation of the right common iliac artery, this ultimately results in an obstruction to venous outflow. Venous spurs are internal venous obstructions consequent to chronic external compression of veins by adjacent structures. Current best practices for the treatment of May-Thurner Syndrome and other non-thrombotic iliac vein lesions are proportional to the severity of the clinical presentation. Leg swelling and pain is best evaluated by vascular specialists, such as vascular surgeons, interventional cardiologists, and interventional radiologists, who both diagnose and treat arterial and venous diseases to ensure that the cause of the extremity pain is evaluated. Diagnosis of MTS/NIVLs is generally confirmed through the use of one or more imaging modalities that may include but is not limited to; Magnetic Resonance Venography, and venogram which, because of the collapsed or flattened left common iliac may not be visible or noticed using conventional venography, this is usually confirmed with intravascular Ultrasound (IVUS). To prevent prolonged swelling or pain as downstream consequences of left common iliac hemostasis, blood flow out of the leg should be improved/increased. Early stage or uncomplicated cases may be managed simply with compression stockings. Late stage or severe May-Thurner syndrome may require thrombolysis if there is recent onset of thrombosis, followed by venoplasty and stenting of the pelvic vein segment after confirming the diagnosis with a venogram and/or intravascular ultrasound. A stent may be used to support the area from further compression following venoplasty.

The current stent options available on the market present with a number of problems including foreshortening, device collapse, device failure, device wear and eventual perforation. Some of the main underlying factors contributing to these problems include a lack of flexibility or too much flexibility. Increased load on the deformation of the stent can cause early fatigue failure, and/or impedance of flow in the overlying iliac artery, potentially causing peripheral arterial disease. The compressed narrowed outflow channel present in

May-Thurner syndrome may cause stasis of the blood, which is an important contributing factor to deep vein thrombosis.

Not every patient having May-Thurner syndrome will experience thrombotic symptoms. Some patients suffering from May-Thurner syndrome may exhibit thrombosis, whilst others may not. Nevertheless, those patients that do not experience thrombotic episodes or symptoms, may still experience thrombosis at any time. If a patient has extensive thrombosis, pharmacologic and/or mechanical (i.e pharmacomechanical) thrombectomy may be necessary. The hemostasis caused by May-Thurner syndrome has been positively linked to an increased incidence of DVT.

The right and left common iliac veins are common locations for deep vein thrombosis, but other locations of occurrence are also common. Non-specific symptoms associated with the condition may include pain, swelling, redness, warmness and engorged superficial veins. Pulmonary embolism, a potential life-threatening complication of deep vein thrombosis, is caused by the detachment of a partial or complete thrombus that travels to the lungs. Deep vein thrombosis can also lead to complications such as chronic venous insufficiency also known as post-thrombotic syndrome (PTS). PTS is another long term complication associated with deep vein thrombosis, which is characterized by pooling of blood, chronic leg swelling, increased pressure, increased pigmentation or discoloration of the skin, and leg ulcers known as venous stasis ulcer.

The term ‘Deep Vein Thrombosis’ (DVT) refers to the formation of blood clots or thrombus within the venous segment, and in itself is not life threatening. However, it may result in life threatening conditions (such as pulmonary embolism) if the thrombus were to be dislodged and embolize to the lungs. Additionally, DVT may lead to loss of venous valvular integrity, life long venous incompetence and deep venous syndrome which includes rest and exercise pain, leg swelling and recurrent risk of DVT and emboli. The following is a non-limiting list of factors that reflect a higher risk of developing DVT including prolonged inactivity, smoking, being dehydrated, being over 60, undergoing cancer treatment and having inflammatory conditions. Anticoagulation which prevents further coagulation but does not act directly on existing clots, is the standard treatment for deep vein thrombosis. Other potentially adjunct, therapies/treatments may include compression stocking, selective movement and/or stretching, inferior vena cava filters, thrombolysis and thrombectomy.

In addition to MTS, NIVL and DVT or Venous Thrombosis compression of any pelvic vein segment; by any cause; on either and or both right or left pelvic veins, can result in changes to venous return. These changes in venous return may have no significant, outwardly visual signs; such as those detailed above for May-Thurner syndrome, Non-thrombotic iliac vein lesions and Deep Vein Thrombosis. These significant and clinical changes however, may manifest in any number of diseases and syndromes including but not limited to hypertension, venous hypertension, hypotension, syncope, orthostatic intolerance, postural orthostatic tachycardia syndrome, atrial fibrillation, heart failure, heart failure with preserved ejection fraction, heart failure with reduced ejection fraction, shortness of breath, shortness of breath on exertion, venous ulcers and erectile dysfunction. Whilst the signs and symptoms of May-Thurner and Deep Vein Thrombosis typically manifest below the compression/occlusion (with the exception of pulmonary edema), the effects detailed here are as a direct and/or indirect association to the reduction in venous return to the heart and the cascade of physiological responses that occur as a result.

Considering the anatomy of the lower extremity venous vasculature, the most likely impingement or restriction of the pelvic veins is due to overriding artery and/or ligament or other structure (example after bowl or pelvic surgery; lymph nodes) against another fixed site such as but not limited to the pelvis or the spine. It may also occur as an impingement of the vein by way of the artery alone such as a vein passing through the space between the internal iliac artery and the common iliac artery.

In any case, treatment of various venous maladies, including those described above can be improved with stents, more specifically venous stents. Better designed, and more specific applications of the various stent features of flexibility, radial force, crush resistance and kink resistance at specific locations along the pelvic venous segments are required to improve outcomes and to prevent further complications as a result of venous stenting for any indication.

The term Iliocaval reduced caliber' refers to iliocaval venous obstruction as detailed above. The obstruction occurs concomitantly with normal arterial vascular aging, reducing arterial elasticity with age. The reduced arterial elasticity results in mechanical external impairment of venous conduction of blood to the heart and subsequent venous congestion. The veins of the iliocaval junction can be entrapped between artery and either bony spine or pelvic structures, ligaments or muscles. This reduction in venous caliber alters normal venous blood flow causing venous congestion, venous hypertension and alterations in the ability of veins to return blood to the heart.

The term ‘pulse transit time’ (PTT) refers herein to the time taken for the pressure wave of each heartbeat to travel between two locations, suitably locations that are pre-determined, for example from the heart to a particular monitored blood vessel, or between two arterial locations. These locations can be referred to as ‘fixed locations’, although the precise location that is monitored may be dependent on the placement of monitoring devices. The fixed locations can be relatively distant from each other, or can be adjacent. In cases where the timing cue relates to an event located in the heart, such as ventricular contraction or aortic valve opening, the PTT is the time elapsing between the timing cue and the detection of the arrival of a wave-front in the monitored blood vessel or remote location. In cases where the timing cue is a different event located in the heart, or is taken to be the time of an event located external to the heart, such as the arrival of a pressure wave in a particular blood vessel, the elapsed time may not correspond to the pressure wave travelling from the heart, and it may be necessary to adjust the elapsed time accordingly. Hence, it will be appreciated that the term ‘fixed’ refers to the choice of the operator to pre-determine the anatomical location or point where sensors are positioned on the subject.

The term ‘pulse wave velocity’ (PWV) refers to the velocity of the pressure wave generated by the contracting heart and a particular blood vessel. It can be calculated from dividing the distance travelled by the pressure wave between two locations by the associated PTT. As above, if the timing cue corresponds to an event located external to the heart, distance can be measured between the locations of the timing cue and the monitored blood vessel. In such cases, it may be necessary to adjust the measured elapsed time, the distance between the two locations, or both, to compensate. For example, if the measured elapsed time corresponds to the difference between the time of wavefront arrival in the carotid and the femoral artery, the real travelled distance of the pressure wave can be estimated by the tape measure distance from the carotid to the femoral artery, multiplied by 0.8, (see Huybrechts et al ‘Carotid to femoral pulse wave velocity: a comparison of real travelled aortic path lengths determined by MRI and superficial measurements’ J Hypertens. 2011 Aug. 29 (8):1577-82, and Bortel et al, ‘Expert consensus document on the measurement of aortic stiffness in daily practice using carotid-femoral pulse wave velocity’ J Hypertens. 2011 Dec. 29 (12):2491).

The term ‘augmentation index’ (Aix) refers to a measure of arterial stiffness derived from the ascending aortic pressure waveform.

The term ‘hydrostatic pressure gradient’ refers to the rate of change in formation of fluid pressure with height, for example, the pressure of a column of blood in a vein related to standing as compared to supine positions. One form of hydrostatic pressure is blood pressure, which is the force exerted upon the walls of blood vessels or chambers of the heart when blood flows through them.

The term ‘arterial stiffness’ refers to a degree of elasticity found within an individual's arteries. Increasing arterial stiffness may occur as a result of aging and atherosclerosis, and is associated with risk of cardiovascular events. PWV increases with arterial stiffness, and due to this relationship, PWV is frequently used to monitor an individual's arterial condition.

The term ‘hypertension’ refers to a chronic medical condition in which the blood pressure in the arteries is persistently elevated above normal levels. The force exerted by the blood is strictly dependent on the resistance of the blood vessels and the cardiac output. Resistant hypertension is defined as uncontrolled blood pressure (BP) despite use of >3 antihypertensive agents from different classes, or controlled blood pressure with the use of >4 hypertensive agents. The UK National Health Service (NHS) defines high blood pressure as systolic pressure (SBP) of above 140 mmHg and a diastolic blood pressure (DBP) of greater than 90 mmHg. The American College of Cardiology/American Heart Association

Task Force defines elevated blood pressure as SBP of above 120 mmHg, with Stage 1 hypertension resulting from SBP of 130-139 or a DBP of 80-89 mm Hg (Hypertension. 2018; 71:e13-e115).

The term ‘atrial fibrillation’ (AF) refers to a heart condition that occurs when electrical impulses fire off from different locations in the atria causing the atria to contract at random.

This reduces the hearts efficiency and results in an abnormal heart rhythm. AF is the most frequent co-morbidity of hypertension, and its development is a marker of increased morbidity and mortality in hypertension, and HFpEF and HFrEF. Atrial fibrillation itself is associated with exertional intolerance, dyspnea, increased congestion—both pulmonary and systemic, risk of stroke and systemic emboli. Ventricular rate control is achieved through the conduction properties of the atrioventricular node.

The terms ‘heart failure with reduced ejection fraction’ (HFrEF) and ‘heart failure with preserved ejection fraction’ (HFpEf) refer to heart failure syndromes associated with excessive sympathetic drive. The excessive sympathetic drive contributes to the morbidity and mortality of heart failure syndromes related to progressive ventricular dysfunction and sympathetic nervous system related cardiac tachyarrhythmias. Ejection fraction is an important measurement in the diagnosis and surveillance of heart failure. Both HFrEF and HFpEF occur when the heart muscle is weakened such that it is unable to consistently pump blood at an adequate rate in response to meet the body's requirement for blood and oxygen characterized by fatigue and shortness of breath. Both HFrEF and HFpEF are associated with reduced exercise tolerance, increased external and resting dyspnea, development of peripheral edema and excessive death due to progressive heart failure as well as sudden death due to arrhythmias.

The term ‘erectile dysfunction’ (ED) (impotence) refers to certain situations when a male cannot initiate or maintain an erection. Proper erectile function requires increasing penile venous engorgement. There can be both physical and psychological causes of erectile dysfunction. Some physical causes include heart disease, occluded blood vessels, high cholesterol and hypertension.

The term ‘syncope (T-LOC)’ (non-neurological or structural) refers to a condition whereby an individual undergoes an abrupt loss of consciousness with a concomitant loss of postural tone. Usually the loss of consciousness is accompanied by falling which is followed by prompt, complete recovery with no intervention required. It is often related to the inability to sustain blood pressure in response to postural changes. The reduced venous return (flow) as the individual becomes orthostatic (changes from sitting/lying to standing) results from a lag in compensatory mechanisms that struggle to keep up.

The term ‘postural orthostatic tachycardia syndrome’ (POTS) refers to a condition which is similar to syncope, but in this instance the blood flow is so poor that during systole the right ventricle walls touch due to an absence of blood (this can be known as empty ventricle syndrome). This results in an incessant tachycardia in an effort to pump more blood into the right ventricle to restore homeostasis. Ultimately this results in the collapse/fainting of the patient as the response is inappropriate to improve blood flow.

The term ‘deep vein thrombosis’ (DVT) refers to a medical condition where a blood clot (thrombus) forms in a vein. This is most commonly found in the leg. One of the major contributing factors to the formation of a clot is the pooling of venous blood. The presence of prolonged venous engorgement and blood flow stasis are conditions of risk for developing deep vein thrombosis. Treatment of the obstructing lesion is critical in relieving the underlying conditions for thrombosis and protecting against recurrence.

The term ‘venous ulcers’ refers to sores that form due to the persistent elevation of venous pressure. Often, they present in association with venous valve regurgitation. They are most commonly found in the lower limbs. It is thought that when venous valves become mechanically blocked or veins become engorged and the valve leaflets cannot co-opt to prevent regurgitation of blood, venous congestion worsens and the hydrostatic forces cause both extravasation of fluid from the veins into interstitium, and activation of inflammatory cytokines. This accumulation of fluid pressure and inflammatory cytokines contributes to skin break down, chronic ulceration and predisposes to local infections.

The term ‘dysmenorrhea’ commonly referred to ‘menstrual cramps’ refers to myometrial contractions of the uterus which is initiated by increased prostaglandins (PGF2) and (PGE2) which result in the cutting off of the supply of oxygen to the muscle tissue of the uterus from nearby blood vessels. This lapse in the supply of oxygen can result in the individual experiencing pain in lower abdomen or pelvic region. Dysmenorrhea can be classified into two categories; primary dysmenorrhea that is not related to any definable pelvic lesion and secondary dysmenorrhea which is related to the presence of pelvic lesions or a pelvic disease e.g. endometriosis, pelvic inflammatory disease, fibroids etc.

The term ‘braided stent’ refers to a metal or metal alloy stent that is produced using a plain weaving technique. The stent comprises a lumen capable of stretching in the longitudinal direction while circumferentially, the multiplicity of filament-like elements intersect a plane that is perpendicular to the longitudinal direction when in the expanded position.

The term ‘kink resistance’ refers to a stent's ability to withstand mechanical loads from the surroundings depending upon the position in the body. Usually, this is based upon the smallest radius of curvature a stent can withstand without the formation of a kink. In areas of high tortuosity within the body it is necessary for a stent to have increased kink resistance to prevent a reduction in lumen patency or even total occlusion.

The term ‘crush resistance’ refers to the ability of a stent experiencing external, non-cardiac, focal or distributed loads to resist collapse. These loads ultimately lead to stent deformation and even full or partial occlusion which can result in adverse clinical consequences.

Without wishing to be bound by theory, the inventors have surprisingly identified that a plurality of significant diseases and symptoms result from venous obstruction or reduced caliber of the iliocaval junction veins in this area thereby impairing an individual's ability to maintain homeostasis. This leads to the progression of several cardiovascular conditions. Furthermore, the inventors have identified that the iliocaval junction is a critically important structure to the maintenance of normal blood flow and contributes to baseline and pathologic sympathetic tone. A restriction of venous return has both upstream and downstream consequences to normal homeostasis that results in the diverse symptomatic responses that manifest in a plurality of previously considered unrelated disease states. Moreover, it has been identified that the implantation of a device within the iliocaval region of the body can alleviate iliocaval venous compression, occlusion, reduction of caliber and/or reduction of venous return which in turn can alleviate the plurality of significant diseases and symptoms that result from venous obstruction or reduced caliber of the iliocaval junction veins in this area.

The inventors have identified that by treating an obstruction and restoring patency (which is defined as visible flow throughout the entire stent system) in the iliocaval region, such as with a device described herein, the symptoms of disease will be reduced. A diagnostic screening protocol combined with a root cause solution to symptom reversal for a multitude of a cascade of related disorders is provided in embodiments of the present invention.

The inventors have recognized that there are established clinical causations that exist today for many or all of the disease states that have been mentioned above. Compression of the iliac segment represents a root cause factor for all of these disease states and has not been described elsewhere, nor has the combination of diagnostic screening and treatment options of iliocaval stenting. The inventors do not suggest that the established mechanisms are in anyway incorrect, rather that compression, change in caliber, or obstruction of the iliac segments should also be considered as a root cause source of disease manifestation. Hence, obstruction of the iliac segments should be considered as part of the screening for both the work up and treatment of current patients with these diseases and additionally utilizing the monitoring tools used for early identification. The invention thereby contributes to prophylactic treatment for individuals who may go on to suffer from a cascade of perk vascular, cardiac and neurological events as a result of undiagnosed and untreated iliocaval compression or obstruction.

It is proposed that one of the reasons the present approach has not been identified before is due to the nature of clinical care. For example, a patient presenting with resistant hypertension will be referred to hypertension experts with no screening for the contribution of the pelvic venous architecture as an underlying cause, similarly, patients with signs of dependent edema will be considered as having underlying ventricular disorders without consideration of pelvic venous anatomy. Furthermore, symptoms of AF will be referred to a cardiologist who will concentrate on the symptoms in relation to the normal functioning of the heart. It may be the case that the patient presenting with AF will experience symptoms such as numbness in the legs but these symptoms will usually be attributed to nothing, or depending on the profile of the patient, linked to unrelated pathology such as old age. However, as demonstrated in the present invention it is possible that the AF and leg numbness are related i.e. the numbness may be a result of an iliocaval venous obstruction. As a consequence, the patient may have developed a downstream challenge causing increased afterload of the LV, leading to hypertension, most likely leading to some mitral valve regurgitation and left atrial enlargement leading to AF progression. Hence, the role of veins as neurohormonally active structure is seldom considered by cardiologists.

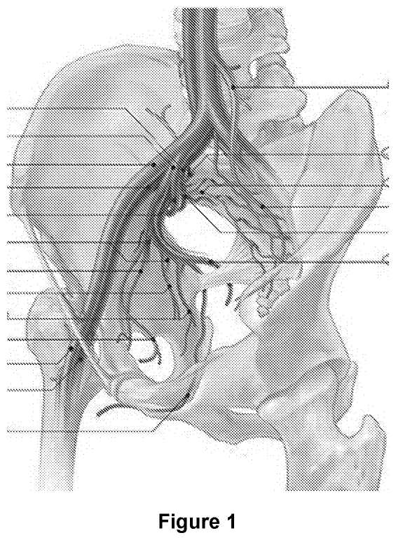

The central veins are systemic veins located in the thorax or abdomen. They differ from the somatic veins and visceral veins. FIG. 1 focusses on the abdomen, the veins are located inferior to the diaphragmatic caval opening at the level of the eighth thoracic vertebra and include the intrahepatic and infrahepatic inferior vena cava (IVC) and the common, external, and internal iliac veins. These veins arise from the confluence and regression of three paired embryonic veins. While the majority of left-sided cardinal veins regress, the right-sided supra and subcardinal veins develop into the inferior vena cava, except for the short hepatic caval segment, which develops from hepatic sinusoids. The entire iliac segment is the region of interest in relation to the present invention which extends from the femoral vein to the lower inferior vena cava and incorporates the external iliac vein, common iliac vein and internal iliac vein.

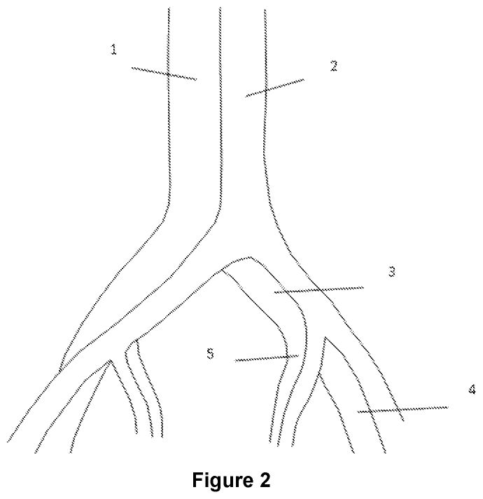

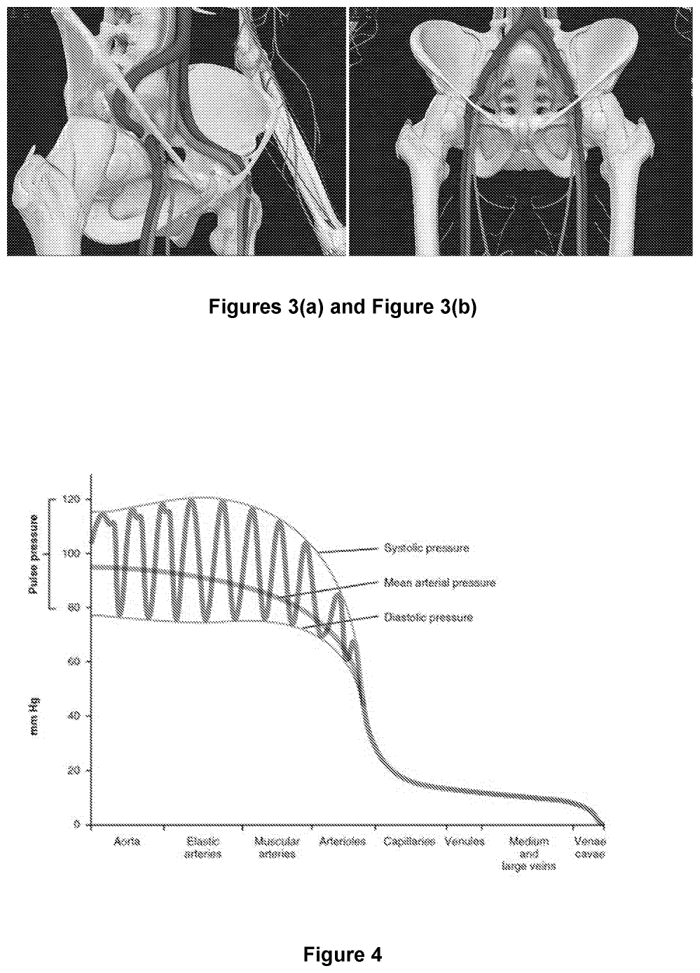

As shown in FIGS. 1 and 2, the iliocaval junction is formed at the point where the internal iliac vein and the external iliac vein unite to form the common iliac vein. The iliocaval junction however, is unsupported as the iliac arteries and veins move over the ischial spine of the pelvis, from the anterior to the posterior pelvis and track up the spinal column (see FIGS. 3(a) and 3(b)). This lack of support, or freedom of movement is important to facilitate ambulatory movement as the torso is bent and rotated, and also through development and growth; internal organ development and through childbirth, for example. The increase in pelvic surgery (commonly hip replacement/repair and pelvic replacement/repair) can displace the natural anatomical position of the iliac arteries and veins. Additionally, increased life longevity as a result of more active lifestyles and improvements in/access to medical therapies and technology puts aging vessels at risk of loss of elasticity, reduced compliance and capacitance.

Anatomical changes in the iliac arteries and veins due to the above-mentioned mechanisms result in changes and adaptations to normal blood flow. These changes are well described in the art. In particular, in reference to venous compression as a result of normal development in otherwise young healthy individuals (particularly woman) resulting in leg edema/leg pain with instant relief following iliac vein stenting. The typical cause being a ligament, bone, muscle or other natural structure occluding the iliac venous segment.

The obstruction to flow can be anywhere along the iliac segment caused by any ligament, muscle, tissue, bony structure, artery, other vein or other structure in that region, that may impinge the vein and cause it to narrow. The reason behind this specific location is that the iliac segment is the only place in the body that the veins are not supported by muscle (like in the legs). Additionally, the veins are not in a straight line and are free to move. Looking at a side perspective of the body, the veins return via the femoral vein from the leg and move from the front of the torso to the rear (spine) crossing the pelvis (bending) and moving up the spine (bending). This unique anatomy does not occur in any other part of the body, making this zone (femoral vein to low IVC) particularly vulnerable to movement and venous compression and obstruction.

Venous obstructions are diverse, obstruction can occur anywhere in the femoral, common and iliac systems and the inferior vena cava. Obstructions can result from the pooling of blood, whereby a clot forms. The treatment of such clots with medication such as anticoagulants (such as heparin) may result in scar tissue deposition inside the vein causing further obstruction which requires treatment to prevent eventual venous occlusion.

Chronic hypertension and persistent increases in arterial pressure are associated with the mechanical loss of elasticity in the arterial walls as a result of natural aging and the contribution of pressure and volume overload to loss of arterial elastic function. Differently, venous congestion, associated venous valvular dysfunction and venous hypertension result in edema and increases of both immune-cytokines and central sympathetic tone which further increases sympathetic activity, blood pressure and the resistance to the hypotensive effect of medications. Venous congestion, itself increases central sympathetic tone. Reciprocally, the reduction of venous congestion and venous hypertension in the lower extremities reduces central tone as demonstrated by the measures of reduced pulse wave velocity and reflection index. Thus, venous hypertension and venous congestion are causes of secondary hypertension and treatment of these occurrences will result in the effectual treatment of a secondary cause of hypertension.

Clinical strategy in the treatment of atrial fibrillation (AF) includes reducing the ventricular rate of patients in atrial fibrillation, preserving underlying sinus rhythm, improving the long term response to treatments (drug and device) to restore sinus rhythm, and reduce the absolute risk of developing atrial fibrillation. Increased sympathetic tone is appreciated as a risk factor for each feature: increased underlying ventricular rate of patients with atrial fibrillation, the risk of reoccurrence after pharmacologic or device treatment of atrial fibrillation, reducing the total atrial fibrillation burden of patients with intermittent atrial fibrillation and reducing the risk of developing the disorder in populations at risk.

Treatment of an underlying cause of adrenergic elevation is expected to reduce the risk of developing atrial fibrillation, the ventricular rates in response to atrial fibrillation and the fibrillation burden in those at risk of intermittent atrial fibrillation. Each of these is expected to improve quality and length of life. Hence, treating a patient with a previously unidentified/recognised venous obstruction results in the reduction of the sympathetically mediated afterload, thereby improving ventricular function and reducing systemic congestion and pulmonary pressures.

Currently no treatment is known that demonstrates a clear improvement in the outcomes for HFpEF, particularly where a patient had preserved systolic function. The disease is associated with dysfunction of the left ventricle. Treatment is largely directed to associated conditions such as hypertension and associated conditions such as edema. There are a few pharmacological treatments that are currently under investigation that have shown promising evidence at early stage to suggest their viability to manage the disease, these include aldosterone agonists, metalloproteinase inhibitors and loop diuretics. Hence there is an urgent need to find alternative treatments. When pelvic venous obstruction contributes to the excessive sympathetic activity or symptomatic congestion, treatment is expected to improve signs and symptoms of heart failure with preserved ejection fraction. This diagnosis and treatment strategy is hitherto unrecognized. This is in contrast to HFrEF which occurs when the heart muscle is not adequately able to contract and as a result less oxygen rich blood is circulated around the body. A key indicator for this disease occurs when patients exhibit lower than normal left ventricular ejection fraction on an echocardiogram. Common symptoms of both diseases are fatigue and shortness of breath. Treatment of an underlying and unrecognised etiologic factor of HFpEf and HFrEF, such as previously unidentified/recognised venous obstruction, is expected to simultaneously reduce morbid symptoms and mortal consequences of these diseases.

The causes of erectile dysfunction (ED) are complex and there can be many contributing factors. Smoking, sedentary lifestyle and being overweight are known to contribute to ED through the narrowing of the penis blood vessels, high blood pressure and high cholesterol.

Furthermore, common medications and psychological factors are also known to have an influence. Blood vessels and nerve essentially control the erection, when the brain sends impulses down the nerve pathways to the penis. These impulses induce relaxation in the smooth muscles of the arteries which supply blood to the penis. Greater volumes of blood are able to enter to penis leading to engorgement and erection of the penis. As such when there is reduced pressure in the pelvic region, venous leakage from the penis occurs, which consequently leads to the inability to maintain an erection as too much blood is leaving the penis. Conventional treatments include medication to treat high blood pressure (hypertension) or to lower cholesterol, hormone replacement or weaning from medication that causes impotence as a known side effect. Treatment of venous congestion according to embodiments of the present invention and consequent improvement venous return allows erections to be sustained.

Increase of venous return in response to prolonged standing or exercise also requires augmented venous return, whereby cardiac output is increased in response to the increased return. When the return of blood flow from the lower extremities is obstructed, the ability to increase cardiac output in response to standing or exercise is impaired and blood pressure falls causing under-perfusion of the central nervous system. This leads to common symptoms of dizziness, or more extreme symptoms of momentary loss of consciousness, upon rising from a seated or prone position. Conventional methods of treatment include use of drugs such as beta-blockers, disopyramide, and ephedrine, for example. Other methods such as lilt-training' encourage the patient to train themselves to undergo progressively prolonged periods of upright posture. In the instance of postural tachycardia syndrome (PoTS), similar to syncope only in this case the blood flow return is so poor that the right ventricle walls touch due to an absence of blood (empty ventricle syndrome) resulting in an incessant tachycardia to try and pump more blood into the right ventricle and collapse/faint.

In this instance the problem is in flow of blood and so the resulting tachycardia as a response is inappropriate to restore homeostasis., According to an embodiment of the invention, providing restoration of adequate venous blood flow and pressure to prevent the tachycardia/collapse response seen with POTS sufferers allows normal orthostatic compensatory mechanisms to prevent empty heart syndrome.

More recently, venous obstruction resulting from Deep Vein Thrombosis (DVT) has been treated with both aspiration of the thrombosis and stenting of the occluded venous segment to prevent its collapse. By reducing the diameter and or compliance of the iliac artery (reduced flow and stasis), a choke point is created by which, when perfect conditions are met (for example, dehydration combined with reduced movement during long distance air travel) the development of thrombosis due to blood stasis can occur. It is necessary to treat the obstructing lesions to relieve the underlying conditions for thrombosis. In an embodiment of the invention, clot removal is implemented along with treatments described herein. Furthermore, in the early stages of recovery, the patient may require the placement of an

AV fistula to improve the venous flow that has been restored through any of the vessel patency procedures—e.g. grafting or stenting.

Venous ulcers may result from an increase in venous pressure due to a restriction in flow in the iliac veins. When the compliance or diameter of the iliac artery is reduced, blood may begin to pool in the lower limbs. In situations where the conditions do not lead to DVT, pooling of blood can result in tissue necrosis which leads to the development of venous ulcers. This results from de-oxygenated blood sitting for extended periods of time in the lower limbs, starving the tissues of oxygen. Symptoms originate with itching and/or swelling of the lower limbs sometimes in combination with discoloured or hardened skin in the affected area.

It is common for ulcers to develop on the interior side of the leg above the ankle. Traditional methods of treatment include directing the patient to wear compression stockings for prescribed periods of time to improve blood flow in the affected area. Traditional wound care, including the cleaning and dressing of the wound is also necessary to assist in the wound healing. It is common to prescribe an antibiotic initially to treat any infection of the ulcer; however, this will not assist in the healing of the ulcer until the underlying cause is removed.

The typical time period to treat an ulcer can last 3 to 4 months, during which time the patient may be immobilised for long periods.

According to an embodiment of the invention, treatment of venous ulcers through the placement of a device within all or a part of the iliocaval junction results in a significant reduction in venous congestion. This allows for fresh oxygenated blood to flow through the affected area and symptom reversal should occur in most cases. However, where the level of tissue death/necrosis is advanced there may be a requirement to implement additional topical treatments.

Conventional treatment of dysmenorrhea includes (i) if the pain is mild; the patient taking pain relief with such as aspirin, acetaminophen, ibuprofen, or naproxen. This must be taken prior to the symptoms of dysmenorrhea presenting. A common natural method to relieve the symptoms is by applying thermal energy to the lower abdomen/lower dorsal region, this can be in the form of a heat pad or a hot water bottle. Where the pain associated with dysmenorrhea is more severe and persists over several months a health care professional might prescribe an oral contraceptive pill (OCP) to the patient. This method of treatment has proven to be effective at reducing menstrual pain in particular as the synthetic hormones of the OCP suppress ovulation. The OCP causes the glands in the lining of the uterus to produce less prostaglandin which consequently reduces the uterine blood flow and cramps.

Although this method of treatment has proven effective it is not suitable for individuals wishing to conceive nor is it suitable in combination with some other everyday medicines. It is also not recommended as a permanent solution as sustained use of an OCP has been linked to long-term health problems including an increased risk of heart attack, stroke and blood clots. Furthermore, it has been linked with an increase in the risk of cervical, breast and liver cancer. According to an embodiment of the present invention, reducing venous obstruction in the iliocaval region such as via venous stenting reduces uterine blood flow, thereby alleviating the symptoms of dysmenorrhea.

Furthermore, in general, it has been demonstrated that an early onset of hypertension is more prevalent in females than in males. Females are also at a higher risk of HFpEF, by having abnormal diastolic filling pressures despite normal LV. Moreover, females with HFpEF have a high rate of combined atrial fibrillation. It is speculated that the reasons for these increased risks may be due to changes in the pelvic veins and arteries, particularly the crushing of the IVC. It is speculated that these changes may in some instances be caused by pregnancy and/or during childbirth.

Further complications caused in this area by pregnancy are the increased incidences of venous thromboembolism post partum, where there is a 30% increase. This increase is a result of impedance of blood flow through the IVC during pregnancy that causes blood stasis. Impedance of blood flow through the IVC during pregnancy may cause a cascade of further problems such as low baby weight. Eclampsia, pre-eclampsia, and gestational hypertension may also flow from reduced venous return. Hence, embodiments of the invention relate to the treatment of female patients, suitably post partum, as an identified sub-population.

Embodiments of the invention may also relate to maintaining venous tone weakened by genetic conditions that cause high mobility and stretch in the venous system, and to treating patients having such conditions.

In relation to the underlying factors affecting all of the above mentioned diseases, it is appreciated that an overall improvement in lifestyle through healthier diet, sustained regular exercise and avoiding habits such as smoking and excessive drinking can assist in the early stage treatment and ultimately in the prevention of all of the aforementioned diseases. However, when a disease is in an advanced state the implementation of these lifestyle changes alone will not be sufficient to instigate any improvement in the symptoms of the patient. The embodiments of the present invention provide scope for major savings at the health economic level. The mis-prescribing of drugs would also be significantly reduced through the implementation of a routine screen/diagnosis technique for identification of venous obstruction in the iliocaval region and, where deemed appropriate, subsequent treatment such as through stenting.

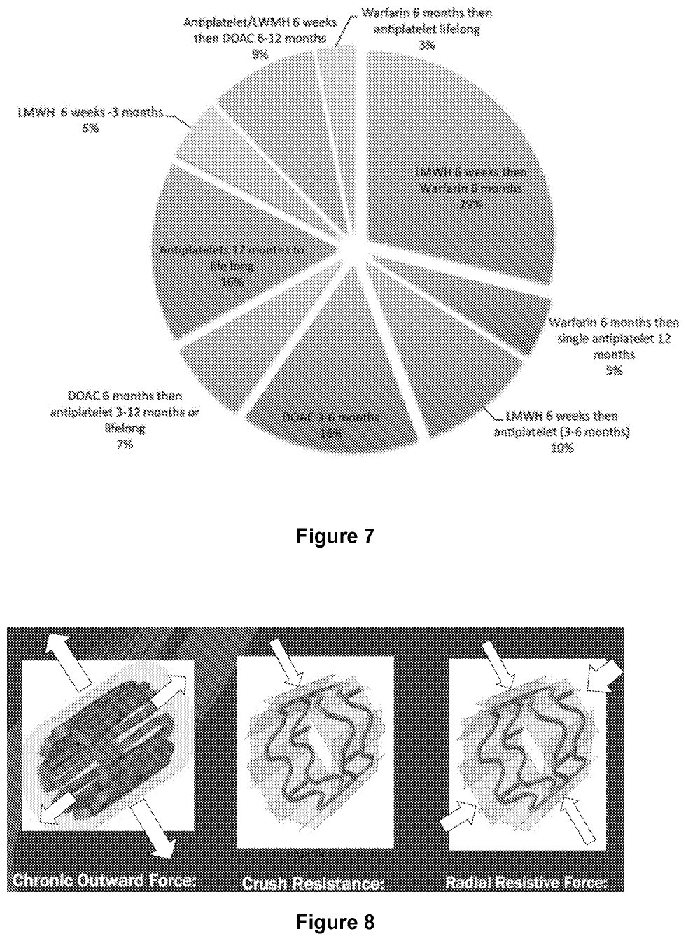

In addition to surgical treatments for obstruction of venous flow in the iliocaval region, such as stenting, AV fistula or venous bypass grafting, administration of drug based therapies may also be appropriate (see FIG. 7). Drugs, such as anti-coagulants, thrombolytics or anti-thrombotic agents may be suitably administered to a patient prior to surgery, following surgery or instead of surgery as appropriate. According to embodiments of the invention, an adult patient having one or more of the diseases and conditions described herein resulting from a thrombus venous obstruction in the iliocaval region may be treated with a dosage regimen of 5000 to 20,000 units sub-cutaneously administered low molecular weight heparin daily for 2 weeks followed by orally administered warfarin 2 to 10 mg daily for 6 months. According to alternative embodiments of the invention, an adult patient (on average 68 kg in body weight) having the diseases and conditions described herein and resulting from a compression/obstruction/caliber reduction without thrombosis and good inflow, may be treated according to a dosage regimen of 5 mg apixaban administered orally daily for 3 months. It will be appreciated that alternative drugs having equivalent or complimentary effects may be prescribed as appropriate. Non-limiting examples of drugs that may be utilised in dosage regimens of the invention for the treatment of one or more of the aforementioned diseases/conditions may include:

Anti-coagulants: heparin; warfarin; fondaparinux; idraparinux; idrabiotaparinux; bivalirudin; dabigatran; agatroban; desirudin; lepirudin; apixaban; rivaroxaban; edoxaban; betrixaban;

Thrombolytics: alteplase; urokinase; reteplase; streptokinase; tenecteplase;

Anti-thrombotics (anti-platelet agents): tirofiban; eptifibatide; abciximab; aspirin; clopidogrel; cilostazol; prasugrel; dipyridamole; ticagrelor; ticlopidine; vorapaxar.

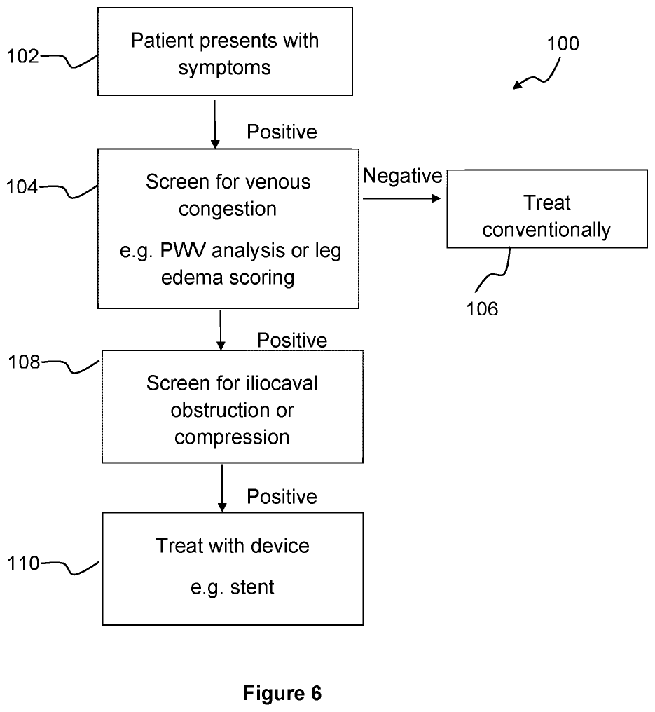

In an embodiment of the invention as described in FIG. 6 there is provided a method of treatment 100 that commences with step 102 in which a patient presenting symptoms indicative of a disease or condition associated with poor venous return. For example, the diseases or conditions may be selected from one or more from the group consisting of: atrial fibrillation; hypertension; erectile dysfunction; venous ulcers; syncope; dysmenorrhea; deep vein thrombosis; and heart failure with preserved ejection fraction or heart failure with reduced ejection fraction. In an initial assessment, at step 104, the individual is subjected to blood pressure monitoring, pulse wave velocity (PWV) analysis, augmentation index, pulse pressure (PP) wave, leg edema scoring including varicose veins and/or exercise stress testing +/− duplex of the pelvic veins. Following the initial assessment the patient is categorized for their risk of iliocaval obstruction/compression. If the patient is considered not to be at risk of iliocaval obstruction/compression, it is recommended that conventional treatments to treat the symptoms of the disease presenting are administered, at step 106. However, if the patient is determined as presenting a high risk of iliocaval obstruction/compression, at step 108 the patient is then screened for iliocaval compression or obstruction using methods including, but not exclusively; Doppler ultrasound of the pelvis and/or venous MRI. If the results from one or more of the above mentioned screening methods or other method not listed indicate that iliocaval compression or obstruction is present or is likely, final confirmation may be sought with invasive diagnostic assessment e.g. contrast venography, intravascular ultrasound, pressure wire and/or fluid or solid state catheter assessment for pressure gradient or compliant balloon pullback through the iliac segment. Upon final confirmation that iliocaval obstruction or compression is present, at step 110 the obstruction or compression can be treated appropriately with a device as described herein (e.g. stent), and optionally with an appropriate pharmaceutical adjunct therapy.

Optionally, the patient can be further monitored for symptomatic improvement for days, months or years following treatment as described above.

In some pathologies it may be appropriate to skip the initial assessment step. This may be due to non-limiting factors such as improved epidemiology, patient history or a patient presenting with a unique combination of symptoms. In this situation, the health care professional would progress immediately to screening for iliocaval compression or obstruction, for example using a medical imaging technique as described herein.

In some embodiments of the invention, a public health screening programme is provided wherein patients meeting a certain age/demographic/lifestyle profile are screened routinely for iliocaval obstruction or compression as a prophylactic measure. In this instance it is envisaged that an individual may be allocated an iliocaval obstruction or compression score, similar to their cholesterol or fasting blood glucose level, that indicates their likelihood of developing one or more of the diseases or conditions described herein. Such an approach would allow for advance treatment of at-risk individuals, according to the methods described herein, before they experience severe morbidity and become a burden on the healthcare system.

In an embodiment the methods of screening for venous congestion include:

1. Measuring arterial stiffness through the use of Pulse Wave Velocity (PWV) analysis and augmentation index analysis:

There are several types of calibrated and certified devices for measuring pulse wave velocity and/or augmentation index both in hospital and in an ambulatory setting (Complior Analyse, Alam Medical, France; SphygmoCor®, AtCor Medical Pty Ltd, Australia). In one such example of a patient in an ambulatory setting, the patient presents with any one of the aforementioned diseases and is monitored using a blood pressure monitoring device. The software is calibrated on a computer and the device is fitted to the patient in accordance with normal usage guidelines. The correct size of cuff is selected to appropriately fit the patient's arm based on the circumference of the upper arm. This ensures accurate and reliable measurement recordings. Typically the cuff is fitted to the patient's upper left arm with the air tube facing in an upward direction. The cuff must be correctly aligned with the patient's brachial artery. Once the device has been fitted correctly, the air flow tube should then be draped around the dorsal side of the neck and connected to the appropriate monitoring device. The device is then used to record PWV in the patient. Additionally the device will typically monitor various other parameters including central blood pressure, augmentation index and central pulse pressure. The patient is typically recorded for a period of up to 24 hours. Statistical analysis methods are subsequently employed to interrogate the data collected and determine whether a statistically significant difference exists. PVVV analysis is believed to have a very strong positive predictive value for the diagnosis of venous congestion (ppv 88.9%). In embodiments of the invention, in the age bracket 50+ the PWV value is around 8.03+/−1.43 for healthy patients and 8.82+/−1.65 for patients with venous congestion. As the measurement varies with age, a typical PWV value for a younger healthy patient (around 30 years) is around 6.81, so a PWV significantly above this value would be indicative of increased arterial stiffness.

In accordance with embodiments of the present invention, a finding of venous congestion may be an initial sign of an asymptomatic development of an iliocaval venous compression, occlusion or reduced caliber venous occlusion occurring/developing.

2. Leg edema scoring—the patient undergoes a physical examination whereby the skins is subjected to applied pressure and ‘pits’ form at the site in question. This examination is usually carried out manually on the patient's shins, ankles and feet. The grading is scaled from 1 to 4 based on the depth of the ‘pit’ that forms and how long it remains before restoration to the original level. At stage 4, pitting is the most severe with ‘pits’ forming at a depth of 8 mm or more, the indentation may remain for more than 2 minutes.

Other methods of monitoring include blood pressure, cardiac monitoring and symptomatology

Screening for iliocaval venous compression, occlusion or reduced caliber, depends upon the severity of the iliocaval venous compression, occlusion or reduced caliber and degree of restriction to flow. This step is usually preceded by screening for venous congestion but in some instances depending on the way in which the patient is presenting with the symptoms, this can be the first screening protocol carried out in order to achieve a swifter diagnosis and treatment.

Confirmation of monitoring data, without the presence of symptoms requires confirmation of the presence/absence of a developed or developing iliocaval venous compression, occlusion or reduced caliber venous occlusion in both iliac vein segments (left and right side). In the instance of elevated blood pressure levels following lifestyle and exercise modification and exclusion of ‘white coat’ hypertension (acute hypertension caused by anxiety in response to clinical monitoring), individuals should be diagnostically screened to rule out the presence of an iliocaval venous compression, occlusion or reduced caliber as part of the standard screening protocols for excluding secondary causes of hypertension.

1. Doppler Ultrasound of the Pelvis

This method uses ultrasound scanning or sonography. A patient is placed in a supine or seated position on an examination table; the patient may be tilted accordingly to manipulate the quality of the ultrasound image. High frequency ultrasound is transmitted through the body via the gel-probe apparatus. The method involves the use of a hand-held ultrasound transducer being placed directly on the patient's skin in the pelvic/groin area. The transducer is then pressed against the skin which is coated with a layer of ultrasound gel to facilitate contact and positioning. The transducer is moved back and forth across the area of interest until a sufficient quality and quantity of images have been captured. The presence of any compression or occlusion of the iliac vein can be indicative of venous congestion. Compression or occlusion may manifest as:
a. Venous engorgement in the iliocaval vessels
b. Presence of collateral venous flow in the iliocaval vessels
c. Presence of venous spurs in the iliocaval vessels

2. Magnetic Resonance Imaging of the Vein (MRI)

This method presents a further non-invasive diagnostic imaging approach that permits visualisation of the soft tissues of the body. An MRI image can detect obstructions and occlusions of the blood vessels within the iliocaval region, as well as venous engorgement, contralateral venous flow and venous spurs. It is common for a patient to be injected intravenously with a contrast agent in order to improve the definition of the veins in the MRI image.

3. Computerised Tomography (CT) Scan

This method combines a series of x-ray images take at various points around the body from multiple angles. These images are then processed via computer to create cross-sectional images (slices) of the bones, soft tissues and blood vessels being examined. CT scans are compatible almost anywhere on the human anatomy. The method allows for a fast and accurate method of examination whilst being pain free to the patient. The patient is placed in a supine position on the examination table. The table is then passed slowly through a tunnel in the scanner, allowing the x-rays to rotate around the body. CT scans of the iliocaval region can detect both venous compression and obstruction.

Invasive diagnostic assessment should be sought as the final confirmation with any of the following methods:

Contrast Venography—wherein a catheter is inserted into the patient in the groin region and navigated to the appropriate position along the iliac segment. The catheter continuously injects fluoroscopy dye to the area of interest in the iliac segment and an x-ray is recorded in real time.

Intra-Vascular Ultrasound—wherein a catheter with a miniaturized ultrasound probe comprised within the distal terminus is inserted into the iliac segment while the proximal terminus of the catheter is attached to computerised ultrasound equipment. This method allows the health care professional to examine patency of the vein from within the blood vessel using imaging ultrasound. Alternatively, a catheter delivers compliant balloon pullback through the iliac segments of the iliocaval vessels can be used to identify presence of fixed venous segments.

In alternative embodiments of the invention, diagnostic procedures may be replaced with or supplemented by appropriate blood work testing. Measuring the level of one or more circulating cytokines over time can represent a biomarker of potential venous occlusion or constriction, particularly in or around the iliocaval region. Chronic elevation of venous pressure predisposes the extravasation of fluid into the interstitial space, activating the release of immunocytokines, which themselves contribute to cardiovascular inflammatory risk. Cytokines that exhibit increased expression include Interleukin-6 (IL-6) and chemokine ligand 2 (CCL2). Other cytokine biomarkers implicated in cardiovascular inflammatory risk may include interleukin-5 (IL-5), tumour necrosis factor-α (TNF-α), endothelin-1, angiotensin II (A-II), endothelin-1 (ET-1), vascular cell adhesion molecule-1 (VCAM-1), and chemokine (C-X-C motif) ligand 2 (CXCL2) and matrix metalloproteinase-2 and/or -9 (MMP-2 and MMP-9). In specific embodiments of the invention, diagnosis of elevated IL-6 levels in excess of around 1.8 μg/mL, suitably at least about 2.0 μg/mL or above, of venous blood may be indicative of venous congestion. Hence, embodiments of the invention include methods for treating the diseases and conditions as described herein, in combination with a companion diagnostic test that identifies the presence of one or more circulating cytokines above a given threshold level that are associated with or indicative of a venous occlusion.

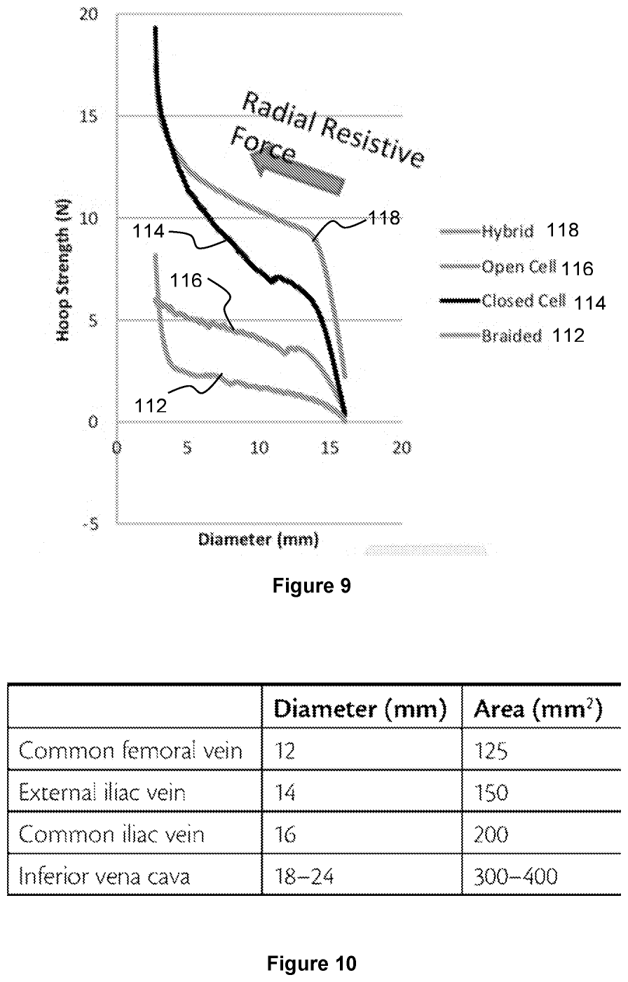

It is desirable for the stent to have a balance of key properties specific to its placement within the body including; crush resistance, flexibility, durability, chronic outward force and minimal foreshortening. A venous stent that can be used to resolve mechanical impingement or kinking of a vein, should have relatively high hoop strength also referred to as radial force or radial compressive strength (hereafter simply referred to as ‘compressive strength’), be self-expanding, have minimal foreshortening and have regions or zones of lower compressive strength and more flexibility. FIG. 9 shows the relationship between hoop strength and diameter for different stent designs.

The region of high compressive strength (HCS) would optimally be bounded by the regions of lower compressive strength (LCS) including the termini of the stent to prevent changes in flow velocity. These properties can be specified for the iliocaval region as shown in Table 1. The use of HCS/LCS sections of stents for the use in adding HCS sections to increase radial force has been detailed previously. However, the use of HCS/LCS combinations for the use of preventing flow velocity changes in the fluid passing through the stent has not been detailed previously.

In embodiments of the invention, there is provided at least one HCS zone with increased radial force that conforms with the correct parts of body that require reinforcement and at least one LCS zone with increased flexibility that allows for kink resistance and accommodates tortuous anatomy. Devices in which there are multiple HCS and LCS zones depending on the requirement of the subject, are encompassed within embodiments of the invention.

TABLE 1

| | |
|---|---|
| Femoral Vein | Flexibility |
| | Kink Resistance |
| | Crush Resistance |
| External Iliac Vein | Flexibility |
| | Kink Resistance |
| Common Iliac Vein | Radial Force |
| | Crush Resistant |
| Inferior vena cava | Radial Force |

Optimised stent designs demonstrate some or all of the following features:
1. Flexible under the inguinal ligament (femoral vein to external iliac vein) but not as a trade-off to crush resistance—strong enough to prevent stent crush, but flexible enough to not impinge movement, in the hip joint or break the stent. Natural movement during walking may crush this area, so resilience of the stent in response to crushing is also desirable. This can be achieved by adopting an open cell stent design to allow for flexibility.
2. Flexibility as the stent transitions from external iliac vein (EIV) to common Iliac vein (CIV) as the iliac vein segment moves over the pelvis. This can be achieved by adopting an open cell stent design to allow for flexibility.
3. As the stent transitions cranially through the CIV and up towards the low inferior vena cava (IVC) it is desirable to increase the radial strength of the stent as this is likely site of compression syndromes such as May-Thurner and other Non-thrombotic Iliac Vein Lesions (NIVLs) and where the Iliac vein begins to rotate and move cranially along the spine. In this region, greater radial force and/or a more closed cell and/or a tighter weave pattern or other stent reinforcing design element would be desirable.
4. At the IVC junction, again, it is desirable for the stent to exhibit increased radial strength as flexibility is less critical in this region. Consequently a more closed cell, tighter weave design is favoured.

In embodiments of the invention, the device, such as a stent, may include one or more self-expanding portions, and one or more portions which are expandable by deformation, for example using a balloon catheter. In certain such embodiments, portions of the stent may include a mesh with a low winding density or high window size, while the terminus portions of the stent include a mesh with a higher winding density or lower window size, the mesh being generally tubular to define a pathway for fluid flow through the centre of the mesh.

Tubes or sheets may be cut to form strut or cell patterns, struts being the parts of the tube or sheet left after cutting and cells or perforations or windows being the parts cut away. A tube (e.g., hypotube) may be cut directly, or a sheet may be cut and then rolled into a tube. The tube or sheet may be shape set before or after cutting.

In one embodiment of the invention, stenting of substantially the entire iliac segment is provided such that an obstruction or compression of the vein is alleviated. This provides protection against future impingements and also provides a stent that is designed specifically for the anatomical location where it will be placed. In creating a single, optimally designed stent with various lengths and sizes and the appropriate properties as shown in FIG. 8, including chronic outward force (radial force that a stent exerts at its expansion), crush resistance and radial resistive forces the potential hazards associated with stenting this region are minimized therefore resulting in a decrease in patient injury and an increase in stent lifespan.

Although the stents described in the embodiments below are illustrated as single stents having a single elongate lumen, it will be appreciated that the different zones or sections of the stent may be formed and installed individually, and subsequently joined or overlapped to form the device. Thus, the term ‘device’ as used herein relates a segmented stent having more than one constituent stent part having a different property and being installed adjacent to or overlapping with another stent part. Stent joining mechanisms will be familiar to the skilled person.

Hence, according to embodiments of the present invention, a device in the form of a stent is provided that combines the open cell radial resistive force of an open cell stent from external iliac/femoral vein to EIV/CIV transition and which then transitions to a stronger resistive force and crush resistance of a closed cell stent moving cranially from the CIV/EIV transition. In an embodiment the venous specific stent is comprised of a material that provides requisite flexibility under the inguinal ligament but maintains radial force under the various portions of the iliocaval venous segments, in particular at the IVC.

In one embodiment, a venous stent is made by cutting slots or pattern in a solid tube of nitinol, shape memory alloy or other bio-compatible material. By using nitinol a laser cut venous stent could be subsequently heat set over a mandrel to achieve a tapered profile. Discontinuous taper or bulge will exert greater radial force and also have a different pattern of struts, connecting bars or other features to increase radial strength. In an embodiment the cranial terminus of the stent proximal to the IVC can be flared to help with securing the stent and also to dissipate radial force through a transition to normal tissue.

A further embodiment contains at least one zone of higher compressive strength bounded on either side by a zone of lower compressive strength, so as to provide a single stent with

LCS zones proximal to and bounding the termini of the stent. A yet further embodiment contains at least two zones of higher compressive strength bounded on either side by zones of lower compressive strength.

In embodiments of the invention, there is a change in axial diameter from the caudal termini to the cranial terminus, it can be a continual change in diameter such that the stent conforms to a taper (e.g. is broadly frustoconical in shape). Alternatively, it could form a plurality of stepped transitions along the longitudinal axis such that the diameter of the body lumen at the first terminus is greater than that of the lumen at the second terminus (e.g. such that it resembles an extended telescope).

In a further embodiment it may be necessary to have a support/anchoring mechanism for the stent in the IVC. This anchoring mechanism may be a simple pair of arms that deploy firstly in the renal veins before the remainder of the stent is deployed caudally. By introducing a support/anchoring mechanism in the renal veins, the stent will be supported from the effects of gravity and movement to keep it positioned and located appropriately, without the use of excess radial force.

FIG. 11 uses a woven stent design with a HCS area (zone), here labelled ‘B’, formed by tighter weave pattern (an increased density of weave) and an LCS area (zone), here labelled ‘A’, with a more open weave pattern. The same effect and desired outcome may also be achieved using laser cut stents in alternative embodiments. The HCS area may be positioned substantially centrally along the longitudinal axis of the stent bounded on either side with LCS areas of varying length. It will be understood by the skilled person that numerous arrangements are conceivable in order to accommodate variations in local iliocaval anatomy between subjects.

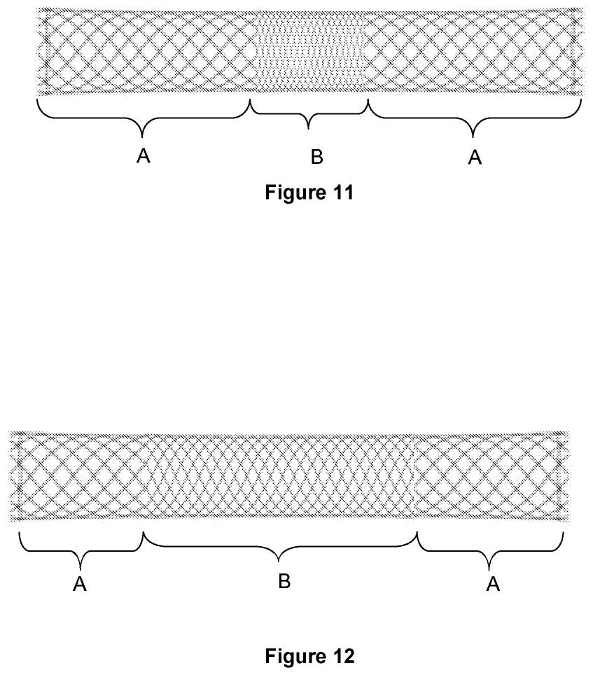

FIG. 12 shows a further embodiment such that the above design may place the HCS area, ‘B’ in the intermediate region of the stent occupying a majority of the stent with LCS zones, ‘A’, on either side. The embodiment of FIG. 12 shows the HCS area to be much longer relative to the LCS zones which extend to the termini. A stent of this design exhibits higher radial crush strength and kink resistance along the majority of its body but possess flexible zones about the termini.

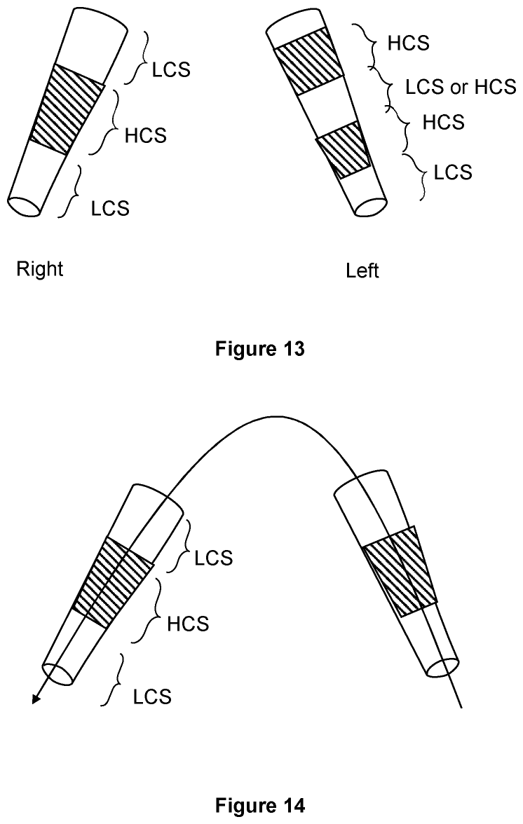

FIG. 13 shows examples of a stent having an asymmetric taper along its length. In FIG. 13, the locations of the HCS and LCS zones are indicated relative to typical impingement or compression locations. In FIG. 13, two stents are shown, for the right and left inferior vena cava respectively.

FIG. 14 provides an example of a long venous stent. It will be appreciated that the full extent of the stent is not depicted, but rather its length is depicted schematically. The stent of FIG. 14 has flexible, tapered ends comprising LCS zones, LCS zones at the centre of the stent, and HCS zones in between the LCS zones.

The stent may be constructed from a variety of different strengths of wire/different weave structures specific to the location of deployment within the common iliac, external iliac, common femoral vein and IVC segments. The stent may comprised of, either separately or in combination, stainless steel, nitinol, cobalt chromium, tantalum, platinum, tungsten, iron, manganese, molybdenum, or other surgically compatible metal or metal alloy. The stent may further comprise non-metal material, including a polymer such as: a bioresorbable material such as poly (1-lactide) (PLLA), polyglycolic acid (PGA), polyglycolic-lactic acid (PLGA), polycaprolactone (PCL), polyorthoesters, polyanhydrides, or another aliphatic polyester fibre material; polypropylene; polyamide; carbon fibre; and glass fibre. In specific embodiments of the invention the stent may comprise both metal and non-metal portions.

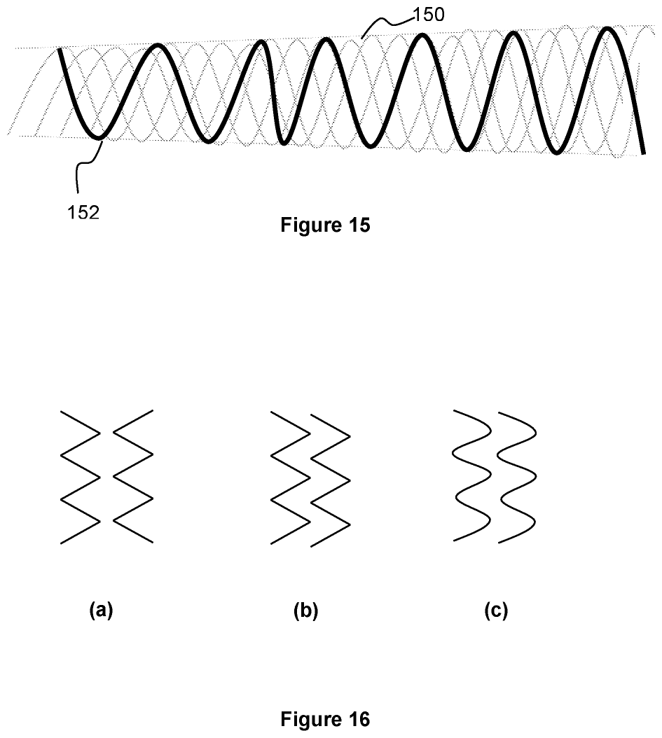

In one embodiment of the present invention as described in FIG. 15, a braided mesh design is provided with a base braided system 150 and additional braid filaments woven into the base braid system at one or more select locations around the circumference of the stent. In FIG. 15, only a single additional braid filament 152 is shown, but it will be appreciated that a plurality of additional filaments may be woven in as desired. These additional braid filaments act as radial compressive strengtheners and enable the stent to have increased crush resistance at specific locations along the stent. The additional braid filaments may be made up of wire that is the same or different to the base braided system. The wire of the additional braid filaments may be flat, round or oval. The tips of the additional braid filaments may be connected to points within the base braid system and/or be free floating termini. The additional braid filaments can adopt either a symmetric or asymmetric distribution around the circumference of the base braided system. The number of additional braid filaments, the type, i.e. material and thickness, of the filaments, the number of windings of the braid filaments, and how they are woven into the base braid system may all be altered to provide a specific, desired amount of crush resistance, in order to meet the requirements of the specific location and characteristics of the structure in which they are to be placed.

In addition to weaving the additional braid filaments into the base braid system generally parallel to the base strands, the filaments can be added in various patterns as shown in FIG. 16 (a) to (c). Common patterns include the zig-zag configuration (also referred to as saw-toothed or z-shaped), shown in (a) and (b) of this Figure, and the sinusoidal configuration (also referred to as s-shaped), shown in example (c). Other patterns may represent hybrid or intermediates between zig zag and sinusoidal patterns. The additional braid filaments may be placed in patterns that are discontinuous but repetitive and may interweave or overlap to provide structural integrity to the device as a whole.

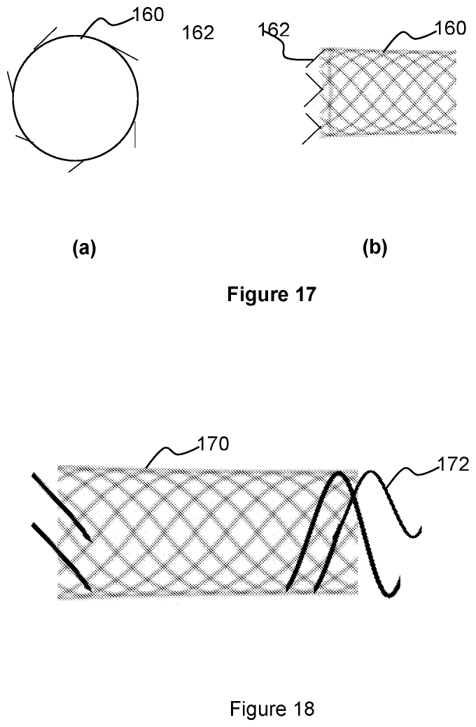

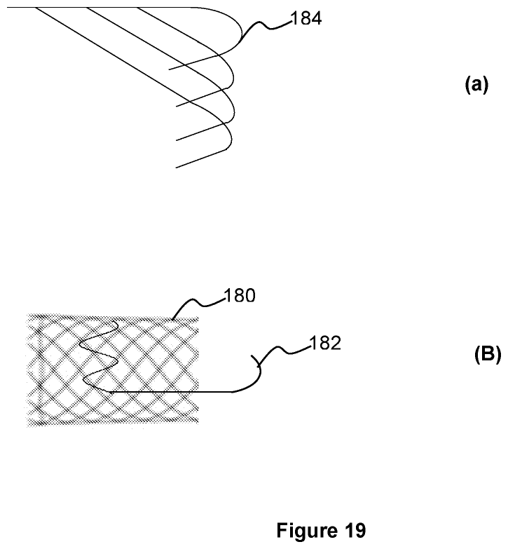

In one embodiment of the present invention as described in FIG. 17, an anchor and/or coupling element 162 is provided at one or both termini of the stent 160 into tissue to prevent migration. This anchor and/or coupling element 162 can serve to couple one or both termini of a venous stent to another venous stent or to another implanted structure. In order to accomplish anchoring or coupling, the anchor may be delivered to the stent along a catheter. As shown in the two examples (a) and (b) of FIG. 19, anchor/coupling elements may be formed of hooks 182 and anchor points 184 from termini of the base braid and/or additional filaments of similar material or different thickness, profile or material. The anchor coupling elements may be comprised of radiopaque material attached to the base braid system with a weld or the use of an adhesive that may comprise additional radiopaque material. As shown in FIG. 18, anchor/coupling elements 172 may extend into the base braid system 170 to provide radial reinforcement.

Changes in flow velocity have been associated with venous stenosis formation within pelvic veins, when the transition zone between the stent and healthy tissue is too great. The goal of the LCS zones at the termini of the venous stents is then to mimic as much as possible the native, healthy venous tissue and to avoid over stretching the tissue thus creating a smoother transition from stent to tissue.

Optionally, the stent may comprise one or more radiopaque markers placed longitudinally and/or radially for visualising the stent and placement. Suitable radiopaque material may include: titanium, tantalum, rhenium, bismuth, silver, gold, platinum, iridium, and tungsten.

The use of radiopaque markers may assist the stent in being placed in the correct rotational orientation towards the adjacent iliac artery if arterio-venous fistula creation is required in addition to simply removing a compression or occlusion. Typically the stent will demonstrate minimal foreshortening on deployment.

In specific embodiments of the invention, the stent may contain a window or cell of increased size and identified by radiopaque markers to allow for the creation of an AV shunting device, without requiring the perforation of the stent base structure. In a specific embodiment the stent or portions of the stent may be covered. Such covering material may include: PTFE; e-PTFE; polyurethane; silicone; papyrus; Dacron®; Gore-Tex®; other polymeric membrane; polyhedral oligomeric silsesquioxane and poly(carbonate-urea) urethane (POSS-PCU); other biodegradable nanofibers.

In specific embodiments of the invention the stent may comprise of a drug coating or combination of drug coating and graft covering to promote re-endothelization; improve endothelial function; reduce inflammatory reaction; inhibit neo-intimal hyperplasia; prevent adverse events such as in-stent restenosis and stent thrombosis through antithrombotic action of heparin.

In embodiments the stent is between 6 and 8 French in diameter and can be deployed through a 10 French introducer catheter device. Typically stents conforming to these parameters are suitable to restore normal luminal diameters in the iliocaval region. With a tapered stent with these sizes are minimal diameters which can be increased diameter in 2 mm increments up to a maximum of 24 mm at the IVC. In embodiments of the invention stent lengths may vary in two ways—firstly in the flexible component from Femoral vein to EIV/CIV transition and the length of the closed cell, strong section in the CIV transition to IVC.

In one embodiment, the invention incorporates a venous specific stent to treat the aforementioned range of diseases. The stent is appropriately sized, positioned and post dilated in accordance with the symptoms of the disease. The stent is inserted into the patient via the iliac segment at the transition from the external iliac vein to common iliac vein—the iliocaval junction.

In an embodiment the venous specific stent is self-expandable on a delivery system that permits for both the slow controlled release and the fast smooth release of the stent depending on the conditions required. This can arise when in a first process of the release the stent is appropriately positioned in the patient and subsequently the stent is then adjusted before deploying the rest of the stent. In an embodiment, the minimum length of the stent is at least 10 cm, typically 15 cm, optionally 18 cm; and maximum length of the stent is at most 28 cm, typically 25 cm and optionally 22 cm. In some embodiments, the stent may be manufactured to a shape and configuration that is desired in the expanded state, and may be compressible so as to fit inside a sleeve for transport on a catheter to a vascular site within the iliocaval region. In one embodiment of the invention, to deploy and expand the stent, the sleeve is drawn back from the stent to allow the shape memory material within the device to return to the pre-set shape, which can anchor the stent in the passages, and which may dilate the passages to reduce occlusion if the stent has sufficient radial strength. Whilst the use of a balloon catheter is not required to expand a fully self-expanding stent, it may be used, in certain instances to improve or optimize the deployment.

The optimal stent sizes in the common iliac and common femoral vein segments are between 16 mm and 12 mm luminal diameters respectively. In an embodiment, as described in FIG. 10, the stent is provided in various sizes or diameter and length. Suitable diameters once deployed range from at least 8 mm, suitably at least 10 mm, typically at least 12 mm and potentially 14 mm. Suitable diameters once deployed range from at most 16 mm, suitably at most 18 mm, typically at most 20 mm diameter and potentially 22 mm.

U.S. Pat. No. 8,257,265 B1 describes the steps of (a) evaluating external symptoms (b) performing IVUS (c) identifying venous lesion and (d) IVUS prior to other CVD diagnostics. In a further embodiment of the invention it is possible to combine any of the stent devices described herein with the use diagnostic imaging of intravascular ultrasound to accurately delivery a stent device to the desired location within the iliocaval region using fluoroscopic guidance; wherein once the venous compression area is confirmed with IVUS and/or fluoroscopy, a special venous stent with a lumen of sufficient size to permit the IVUS catheter to be placed inside is advanced over a wire.

In a further embodiment, the IVUS catheter is between 3 and 4 French in size at the transducer over a guidewire of a size compatible with the corresponding IVUS catheter, typically at least 0.25 mm (0.010 inches).

In a particular embodiment the present invention provides for a method for treating venous compression comprising the steps of:

I. Gross anatomic evaluation with a simultaneous and/or selective arterial and venous contrast bolus to identify points of interest for venous compression.

II. Identifying with intravascular ultrasound (IVUS) catheter to identify an area of venous compression (VC) or multiple,

III. Using IVUS to identify areas of normal vessel proximal and distal to the venous compression or compressions

IV. Placing a venous stent co-axial to the IVUS catheter

V. Using IVUS and fluoroscopy to locate the venous stent

VI. Treating the venous compression by placing the venous stent in the vein lumen.

In a particular embodiment the present invention provides for a device for treating venous compression using co-axial intra-vascular ultrasound comprising;

I. A venous stent; self-expanding

II. Pre-loaded into a delivery catheter, through which an IVUS catheter can be passed and through which a guidewire can be passed.

In a particular embodiment, the present invention provides a self-expanding venous stent with at least one area of HCS by an area of LCS on at least one side through which a guidewire may be passed.

In a particular embodiment, the present invention provides a self-expanding venous stent with at least one area of HCS by an area of LCS on at least one side through which an IVUS catheter and guidewire may be passed as a co-axial system; wherein the self-expanding venous stent delivery IVUS catheter and guidewire of the coaxial system are all slidably disposed to allow the IVUS catheter to remain stationary at the first location in a vein to be treated, while the venous stent is slidably positioned at a second location and the venous stent delivered; wherein the system allows for the fluoroscopic identification of both the IVUS and venous stent and which permits the injection of contrast agent through the stent catheter while the IVUS catheter is in coaxial position.

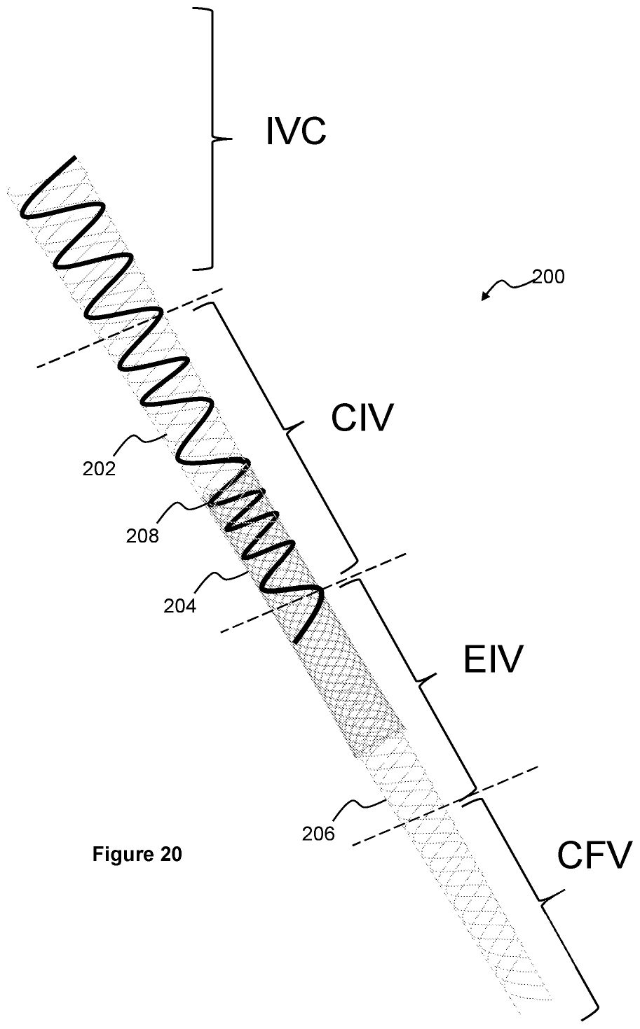

FIG. 20 brings together the elements described above to illustrate how the iliocaval region may be stented. Shown in FIG. 20 is an example stent 200, comprising either a single stent or a plurality of adjacent or joined stents. The different vessels of the iliocaval region for which the parts of the stent of FIG. 20 are intended are indicated using brackets. The transitions between vessels are indicated on the stent using dotted lines.

The stent of FIG. 20 comprises three sections: a first section 202 for placement in the IVC and CIV, a second section 204 for placement in the CIV and EIV, and a third section 206 for placement in the EIV and the femoral vein (CFV). The first section and half of the second section form a zone of high compression at the superior end of the stent. This transitions to a zone of low compression in the other half of the second section and third section, for placement in the EIV and the CFV. As described above, it is necessary to implement a stent structure having crush resistance in the IVC and CIV, kink resistance and flexibility in the EIV, and flexibility so as not to impede movement in the CFV, and this is achieved by the use of additional braid filaments to change the properties of the base stent at different points along its length.

The high radial force for the IVC is achieved in the first section by weaving an additional braid filament 208 into the base braid of the stent. The crush resistance required in the region of the CIV is achieved with the more tapered part of the first section 202 with the additional braid filament 208 and the first half of the second section 204, which has a tighter weave, combined with the additional braid filament 208.

By allowing the additional braid filament 208 to only extend part of the way along the second section 204, the properties of the second section 204 change, so that the second half of the second section 204 is suitable for the EIV. Therefore, the kink resistance and flexibility for the EIV is achieved using the tight weave of the second section 204. The first half of the third section 206 is also suited to the EIV, which has a more open weave but higher flexibility. Finally, the end of the third section 206 also has a more open weave and a smaller diameter, which is particularly resilient to being crushed and has a low fracture rate, meaning it is therefore suitable for the CFV.

The stent is formed from braided nitinol along its length to improve flexibility.

As can also be seen, the stent tapers towards inferior end, to match the diameter of the vessel. Typically, the diameter of the stent at the IVC, i.e. the superior, end will be between 18-24 mm as indicated in FIG. 10. The diameter tapers along the length of the stent according to the measurements indicated in FIG. 10, so that the stent at the inferior end, which is to be located in the CFV, has a diameter of between approximately 12 and 14 mm.

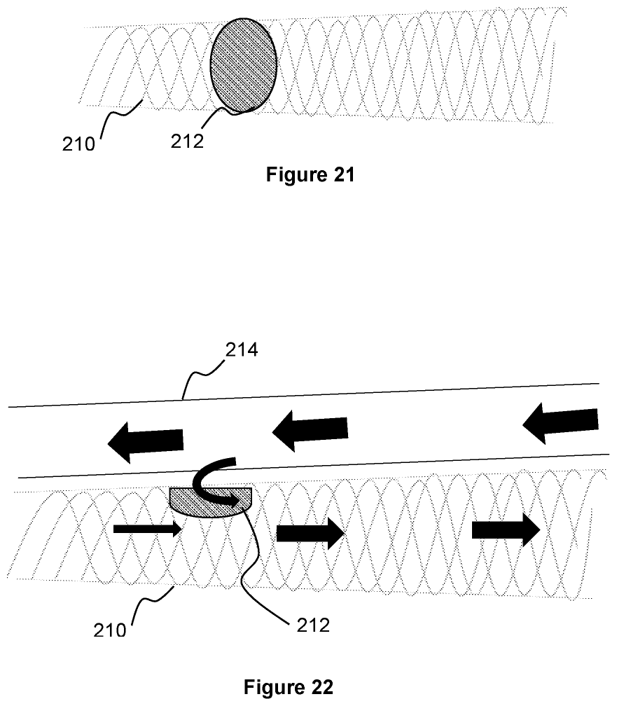

FIG. 21 shows an example of a stent 210 for use in the CFV and EIV, where the artery and vein are close together. This makes this portion of the vessel highly suited to the formation of an AV fistula. The formation of a fistula, as noted above can be provided by providing a window in the stent. In addition, or alternatively, an inflow booster tract 212 may be provided in the stent to aid with the formation of an AV fistula. The tract is formed of a permeable material, such as Dacron.

FIG. 22 shows the stent 210 of FIG. 21 in position within a vein (not shown for clarity), with the tract 212 oriented for formation of a fistula facing the artery 214. Once an AV fistula is formed, blood flows as illustrated by the arrows, through the tract and into the vein from the artery.

### EXAMPLES

### Example 1

### Use of Cytokine Biomarkers as Indicators of Vascular Congestion

In a trial conducted in dialysis using unilateral tourniquet inflation and and varying degrees of venous stenosis (European Heart Journal, Volume 35, Issue 7, 14 Feb. 2014, Pages 448-454) the levels of cytokines (specifically; plasma interleukin-5, endothelin-1, angiotensin II, vascular cell adhesion model and chemokine ligand) in both the fistula arm and the non-fistula arm in the same patient were examined. It was found that there was a difference in level of measures cytokines when comparing the fistula arm with no stenosis against the non-fistula arm, there was no difference in cytokine levels. When the patient began to develop a stenosis varying degrees, the difference in cytokines began to change. The fistula arm exhibited higher levels of cytokines than the non-fistula arm.

Measuring the level of circulating cytokines over time was shown to be a non-specific biomarker (other factors can cause cytokine levels to increase) that can provide an indication that further assessment is required to look for the presence/development of an compressed iliac segment.

### Example 2

### Clinical Presentation of Hypertensive Patient with Occlusion of the Iliocaval Region

FIGS. 3(a) and 3(b) show different views of the pelvic region. The pelvis is connected to several blood vessels. Both the internal iliac artery and the external iliac artery arise in the sacro-iliac joint, they meeting at the iliac junction. It can be seen that the venous system while posterior follows an almost identical path to that of the anterior arterial system. The venous system is responsible for draining deoxygenated blood and returning it to the heart. It can be seen that when the femoral vein crosses underneath the inguinal ligament it becomes the external iliac vein. The vein runs along the medical aspect of the external iliac artery, it then joins with the internal iliac vein to form the common iliac vein. The majority of the venous drainage in the pelvic region occurs through the interior iliac vein. The vein receives numerous tributaries including but not limited to the obturator vein, the vesical veins and the gluteal veins.

The human body has an array of blood vessels that take oxygenated blood away from the heart (arteries) and back to the heart for re-oxygenation via the lungs (veins). This flow of blood around this circuit is controlled via a pressure regulation system whereby blood flows run down a pressure gradient curve from high pressure (LV/Aorta—normal 120/80 mmHg) to low pressure (RA 12/0 mmHg) for refilling of the circuit. From FIG. 4 it can be seen that the pulse pressure is the difference between the systolic and diastolic pressures.

Intravascular pressure is regulated through maintenance of sympathetic tone. Sympathetic regulation is the activation of muscle fibres in the arterial system to allow for vessel dilation and active constriction to maintain these arterial tone and pressure gradients to facilitate free blood flow. Differently, both venous capacitance and tone are regulated through sympathetically mediated venous smooth muscle cells. Contraction reduces capacitance and raises venous pressure. Moreover, veins have series of valves to prevent retrograde flow of dependent blood due to the hydrostatic pressure gradient.

The patient is a 65 year old white male with a height of 170 cm and a weight of 88 kg. His blood pressure was recorded at 142/72 and his ambulatory blood pressure was recorded at 137/74. He has suffered with hypertension since 1995 and Paroxysmal Atrial Fibrillation (PAF) since 2010 for which he underwent ablation therapy in 2013. He has had pain in his upper left leg since 2014 with no diagnosis available and hypercholesteremia since 2012. In order to control his blood pressure he had been prescribed 3 anti-hypertensive medications; Indapamide (2.5 mg), Olmesartan (10 mg) and Felodipine (10 mg). However his condition remained refractory to medical treatment.

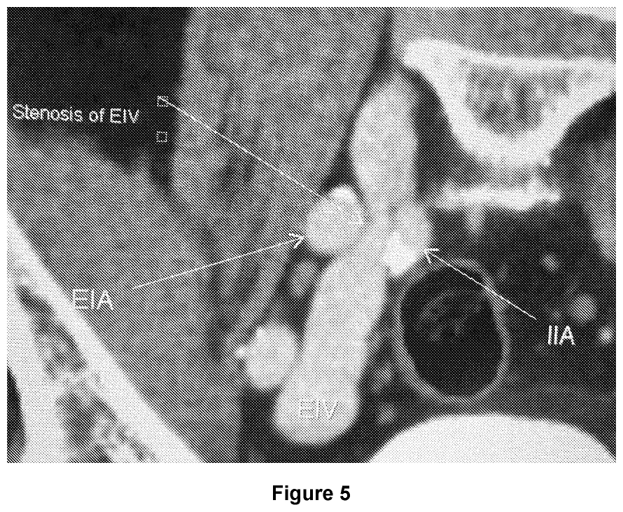

In FIG. 5, the MRI image shows the patient has suffered a previously undiagnosed compression of >60% of the External Iliac Vein (EIV) caused by the External Iliac Artery (EIA) and the Internal Iliac Artery (IIA). Treatment to address this compression is proposed to alleviate the symptoms of hypertension experienced by the patient.

1. A self-expanding stent device comprising:
a woven or braided elongated body that defines a lumen within, the body having at least a first and at least a second terminus and a longitudinal axis located therebetween;
wherein the body comprises at least a first zone and at least a second zone along the longitudinal axis;
wherein when the device is in an expanded configuration the first zone has a first radial strength that is resistant to an external compressive force, and the second zone has a second radial strength that is resistant to an external compressive force, wherein the radial strength of the first zone is greater than that of the second zone; and
wherein the diameter of the body lumen proximal to the first terminus is greater than that of the lumen proximal to the second terminus.

2. The device of claim 1, wherein the body is tapered from the at least a first terminus to the at least a second terminus thereby resulting in a reduction in the diameter of the lumen along the longitudinal axis.

3. The device of claim 1, wherein diameter of the first terminus is not greater than 30 mm.

4. The device of claim 1, wherein the diameter of the second terminus is not less than 6 mm.

5. The device of claim 1, wherein the first zone is located substantially centrally along the longitudinal axis.

6. The device of claim 1, wherein the first zone is located proximally to the first terminus.

7. The device of claim 1, wherein the second zone is located proximally to the first terminus.

8. The device of claim 1, wherein the second zone is located proximally to the second terminus.

9. The device of claim 1, wherein the device comprises a plurality of first zones arranged sequentially along the longitudinal axis, and at least one second zone arranged between adjacent first zones.

10. The device of claim 1, wherein the device comprises a plurality of first zones and a plurality of second zones, wherein the arrangement of first and second zones alternates along the longitudinal axis.

11. The device of claim 1, wherein the first and second zones are not of equal length.

12. The device of claim 1, wherein the arrangement of first and second zones conforms to the anatomy of a subject into which the device is to be placed.

13. The device of claim 1, wherein the device is adapted to be deployed within a vein.

14. The device of claim 1, wherein the device is adapted to be deployed within the iliocaval region of a human subject.

15. The device of claim 14, wherein the device is adapted to be deployed within iliocaval region and, when in the expanded configuration, to extend through the iliac vein.

16. The device of claim 14, wherein the device is adapted to be deployed within iliocaval region and, when in the expanded configuration, the first terminus is located within the inferior vena cava and the second terminus is located within the femoral vein.

17. The device of claim 1, wherein stent device comprises either separately or in combination, stainless steel, nitinol, cobalt chromium, tantalum, platinum, tungsten, iron, manganese, molybdenum, or other surgically compatible metal or metal alloy.

18. The device of claim 1, wherein the stent or portions of the stent comprise a covering.

19. The device of claim 18, wherein the covering comprises either separately or in combination, PTFE, e-PTFE, polyurethane, silicone, papyrus, Dacron®, Gore-Tex®, other polymeric membrane, polyhedral oligomeric silsesquioxane and poly(carbonate-urea) urethane (POSS-PCU), or other biodegradable nanofibers.

20-25. (canceled)

26. A system for deployment of a venous stent, the system comprising:
i. a delivery catheter; and
ii. a self-expanding stent as described in claim 1.

27. The system of claim 26, wherein the delivery catheter further comprises a imaging ultrasound transducer (IVUS) capability.

28-38. (canceled)
